SEQUENCE LISTING (3 sequences)

SEQ ID NO: 1 - protein, 456 aa, artificial sequence
  source: Antibody heavy chain
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, artificial sequence
  source: Antibody light chain
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 42 aa, artificial sequence [sequence omitted]

(19) United States
(12) Patent Application Publication
(10) Pub. No.: US 2011/0070225 A1
(43) Pub. Date: Mar. 24, 2011

(54) Beta antibody parenteral formulation

(76) Inventors: Pierre Goldbach, RIixheim (FR); Hanns-Christian Mahler, Basel (CH); Robert Mueller, Basel (CH); Christine Wurth, Loerrach (DE)

(21) Appl. No.: 12/448,190

(22) PCT Filed: Dec. 11, 2007
(86) PCT No.: PCT/EP2007/010825

§ 371 Date: Dec. 22, 2009

(30) Foreign Application Priority Data
Nov. 12, 2006 (EP) 06025590.8

(51) Int. Cl. A61K 39/395; A61P 25/28

(52) U.S. Cl. 424/133.1; 424/172.1

(57) ABSTRACT

The present invention relates to a stable pharmaceutical parenteral formulation of an antibody, antibody molecule, a mixture of antibodies and/or a mixture of antibody molecules against the amyloid-beta peptide (Abeta) and a process for the preparation. Furthermore, corresponding uses are described.

The present invention relates to a stable pharmaceutical parenteral formulation of an antibody, antibody molecule, a mixture of antibodies and/or a mixture of antibody molecules against the amyloid-beta peptide (Abeta) and a process for the preparation thereof. Furthermore, corresponding uses are described.

In a first aspect, the invention relates to a stable pharmaceutical parenteral Abeta antibody pharmaceutical formulation comprising:
about 1 to about 250 mg/mL Abeta antibody;
about 0.001 to about 1% of at least one surfactant;
about 1 to about 100 mM of a buffer;
optionally about 10 to about 500 mM of a stabilizer and/or about 5 to about 500 mM of a tonicity agent;
at a pH of about 4.0 to about 7.0.

In particular, the present invention relates to an Abeta antibody formulation wherein the comprised Abeta antibodies (or mixtures thereof) are capable of specifically binding the amyloid-beta peptide. Antibodies that specifically bind Abeta are known in the art. Specific examples of Abeta antibody that can be used in the formulation according to the invention have been described in the published PCT patent application WO 03/070760 and especially in the claims, the content of which is incorporated herein by reference.

The amyloid-beta peptide, which is also termed “amyloid β”, “Aβ”, “Aβ4” or “β-A4” and, in particular in context of this invention “Abeta”, is a main component of the extracellular neuritic plaques that are associated with amyloidogenic diseases such as Alzheimer's disease; see Selkoe (1994), Ann. Rev. Cell Biol. 10, 373-403, Koo (1999), PNAS Vol. 96, pp. 9989-9990, U.S. Pat. No. 4,666,829 or Glenner (1984), BBRC 12, 1131. This amyloid β is derived from “Alzheimer precursor protein/β-amyloid precursor protein” (APP). APPs are integral membrane glycoproteins (see Sisodia (1992), PNAS Vol. 89, pp. 6075) and are endoproteolytically cleaved within the Abeta sequence by a plasma membrane protease, α-secretase (see Sisodia (1992), loc. cit.). Furthermore, further secretase activity, in particular β-secretase and γ-secretase activity leads to the extracellular release of amyloid-β (Aβ) comprising either 39 amino acids (Aβ39), 40 amino acids (Aβ40), 42 amino acids (Aβ42) or 43 amino acids (Aβ43); see Sinha (1999), PNAS 96, 11094-1053; Price (1998), Science 282, 1078 to 1083; WO 00/72880 or Hardy (1997), TINS 20, 154.

Aβ has several naturally occurring forms, whereby the human forms are referred to as the above mentioned Aβ39, Aβ40, Aβ41, Aβ42 and Aβ43. The most prominent form, Aβ42, has the amino acid sequence (starting from the N-terminus): DAEFRHDSGYEVHHQKLVFFAEDVGSNKGAIIGLMVGGVVIA (SEQ ID NO: 3). In Aβ41, Aβ40, Aβ39, the C-terminal amino acids A, IA and VIA are missing, respectively. In the Aβ43-form an additional threonine residue is comprised at the C-terminus of the above depicted sequence (SEQ ID NO: 3).

Antibody molecules, as part of the group of protein pharmaceuticals, are very susceptible to physical and chemical degradation, such as denaturation and aggregation, deamidation, oxidation and hydrolysis. Protein stability is influenced by the characteristics of the protein itself, e.g. the amino acid sequence, and by external influences, such as temperature, solvent pH, excipients, interfaces, or shear rates. So, it is important to define the optimal formulation conditions to protect the protein against degradation reactions during manufacturing, storage and administration. (Manning, M. C., K. Patel, et al. (1989). “Stability of protein pharmaceuticals.” Pharm Res 6(11): 903-18., Zheng, J. Y. and L. J. Janis (2005). “Influence of pH, buffer species, and storage temperature on physicochemical stability of a humanized monoclonal antibody LA298.” Int_J_Pharm.)

Administration of antibodies via subcutaneous or intramuscular route requires high protein concentration in the final formulation due to the often required high doses and the limited administration volumes. (Shire, S. J., Z. Shahrokh, et al. (2004). “Challenges in the development of high protein concentration formulations.” J Pharm Sci 93(6): 1390-402, Roskos, L. K., C. G. Davis, et al. (2004). “The clinical pharmacology of therapeutic monoclonal antibodies.” Drug Development Research 61(3): 108-120.) The large-scale manufacturing of high protein concentration can be achieved by ultrafiltration processes, drying process, such as lyophilisation or spray-drying, and precipitation processes. (Shire, S. J., Z. Shahrokh, et al. (2004). “Challenges in the development of high protein concentration formulations.” J Pharm Sci 93(6): 1390-402.)

Andya et al. (U.S. Pat. No. 6,267,958, U.S. Pat. No. 6,85,940) describe a stable lyophilized formulation of an antibody, which is reconstituted with a suitable diluent volume to achieve the required concentration. The formulation comprises a lyoprotectant, a buffer and a surfactant.

Liu et al. (Liu, J., M. D. Nguyen, et al. (2005). “Reversible self-association increases the viscosity of a concentrated monoclonal antibody in aqueous solution.” J Pharm Sci 94(9): 1928-40.) examined the viscosity behavior of high concentration antibody formulations. Three monoclonal antibodies, constructed from the identical IgG1 framework, were examined for their self-association at high protein concentration. The three antibodies demonstrated no consistent viscosity-profile and showed significant differences in their self-association behavior.

One object of the present invention is to provide a formulation of an Abeta antibody or of mixtures of such antibodies, which is/are concentrated to the required concentration by reconstitution of a lyophilized formulation with a suitable volume or by removing the solvent by an ultrafiltration process. The formulation demonstrates sufficient stability during manufacturing, storage and administration. As demonstrated by Liu et al., antibodies show an unpredictable viscosity-concentration profile. (Liu, J., M. D. Nguyen, et al. (2005). “Reversible self-association increases the viscosity of a concentrated monoclonal antibody in aqueous solution.” J Pharm Sci 94(9): 1928-40.) In comparison to the patents U.S. Pat. No. 6,267,958 and U.S. Pat. No. 6,685,940 the presented formulation provides equal or better stability of an Abeta human antibody during storage and has a viscosity, which is suitable for the subcutaneous or intramuscular administration route.

Examples of Abeta antibodies that are useful in the present invention are immunoglobulin molecules, e.g. IgG molecules. IgGs are characterized in comprising two heavy and two light chains (illustrated e.g. in FIG. 1) and these molecules comprise two antigen binding sites. Said antigen binding sites comprise “variable regions” consisting of parts of the heavy chains (VH) and parts of the light chains (VL). The antigen-binding sites are formed by the juxtaposition of the VH and VL domains. For general information on antibody molecules or immunoglobulin molecules see also common textbooks, like Abbas “Cellular and Molecular Immunology”, W.B. Sounders Company (2003).

In one embodiment, the parenteral formulation of the present invention comprises Abeta antibody (or mixture of such antibodies) in which in at least one of the variable regions in the heavy chain of said antibodies comprises a N-glycosylation. The glycosylated asparagine (Asn) in the variable region of the heavy chain (VH) may be in the complementarity determining region 2 (CDR2 region), said glycosylated asparagine (Asn) may be on position 52 in the variable region of the heavy chain (VH) as shown in SEQ ID NO: 1.

The term “mono-glycosylated antibody” relates to an antibody molecule comprising an N-glycosylation in one (V_{H})-region of an individual antibody molecule“; see also FIG. 1. The term “double-glycosylation antibody” defines an antibody molecule which is N-glycosylated on both variable regions of the heavy chain” (FIG. 1). Antibody molecules which lack a N-glycosylation on both heavy chain (V_{H})-domains are named “non-glycosylated antibodies” (FIG. 1). The mono-glycosylated antibody, the double-glycosylated antibody and the non-glycosylated antibody may comprise the identical amino acid sequences or different amino acid sequences.

The mono-glycosylated antibody and the double-glycosylated antibody are herein referred to as “glycosylated antibody isoforms”. A purified antibody molecule characterized in that at least one antigen binding site comprises a glycosylation in the variable region of the heavy chain (VH) is a mono-glycosylated antibody which is free of or to a very low extent associated with an isoform selected from a double-glycosylated antibody and a non-glycosylated antibody, i.e. a “purified mono-glycosylated antibody”. A double-glycosylated antibody in context of this invention is free of or to a very low extent associated with an isoform selected from a mono-glycosylated antibody and a non-glycosylated antibody, i.e. a “purified double-glycosylated antibody”.

The formulations according to this invention may contain mono-glycosylated or double-glycosylated or non-glycosylated antibodies, or specifically defined mixtures thereof. The antibody mixtures or antibody pools provided herein may comprise 50% mono-glycosylated and 50% double-glycosylated antibodies as defined herein. However, also envisaged are the ratios of 30/70 to 70/30. Yet, the person skilled in the art is aware that also other ratios are envisaged in the antibody mixtures of this invention. For example, also 10/90 or 90/10, 20/80 or 80/20 as well as 40/60 or 60/40 may be employed in context of this invention. A particular useful ratio in the antibody mixtures comprised in the formulation of the invention comprises double-glycosylated and mono-glycosylated antibody as defined herein above is a ratio from 40/60 to 45/55.

The term “which is free of or to a very low extent” denotes the complete absence of the respective other (glycosylation) isoforms or a presence of another (glycosylated) isoform in a concentration of at the most 10%, e.g. at the most 5%, e.g. at the most 4%, e.g. at the most 3%, e.g. at the most 2%, e.g. at the most 1%, e.g. at the most 0.5%, e.g. at the most 0.3%, e.g. at the most 0.2%.

The term “antibody(ies)” is used herein synonymously with the term “antibody molecule(s)” and comprises, in the context of the present invention, antibody molecule(s) like full immunoglobulin molecules, e.g. IgMs, IgDs, IgEs, IgAs or IgGs, like IgG1, IgG2, IgG2b, IgG3 or IgG4 as well as to parts of such immunoglobulin molecules, like Fab-fragments, Fab′-fragments, F(ab)2-fragements, chimeric F(ab)2 or chimeric Fab' fragments, chimeric Fab-fragments or isolated VH- or CDR-regions (said isolated VH- or CDR-regions being, e.g. to be integrated or engineered in corresponding “framework(s)”) Accordingly, the term “antibody” also comprises known isoforms and modifications of immunoglobulins, like single-chain antibodies or single chain Fv fragments (scAB/scFv) or bispecific antibody constructs, said isoforms and modifications being characterized as comprising at least one glycosylated VH region as defined herein. A specific example of such an isoform or modification may be a sc (single chain) antibody in the format VH-VL or VL-VH, wherein said VH comprises the herein described glycosylation. Also bispecific scFvs are envisaged, e.g. in the format VH-VL-VH-VL, VL-VH-VH-VL, VH-VL-VL-VH. Also comprised in the term “antibody” are diabodies and molecules that comprise an antibody Fc domain as a vehicle attached to at least one antigen binding moiety/peptide, e.g. peptibodies as described in WO 00/24782. It is evident from the above that the present invention also relates to parenteral formulations of Abeta antibodies that comprise “mixtures” of antibodies/antibody molecules. A particular “mixture” of said antibodies is described above, namely a mixture of “mono” and “double”-glycosylated antibodies directed against Abeta.

“Antibody fragments” also comprises such fragments which per se are not able to provide effector functions (ADCC/CDC) but provide this function in a manner according to the invention after being combined with appropriate antibody constant domain(s).

The Abeta antibody(ies) that may be comprised in the inventive formulation(s) are, inter alia, recombinantly produced Abeta antibody(ies). These may be produced in a mammalian cell-culture system, e.g. in CHO cells. Such mammalian cell culture systems are particular useful in the preparation of Abeta antibodies or Abeta antibodies/antibody molecules that are glycosylated like the specific herein exemplified Abeta antibody that comprises a N-glycosylation in the variable region. The antibody molecules may be further purified by a sequence of chromatographic and filtration steps e.g. in order to purify the specific glycosylated antibody isoforms as described herein below.

The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M.S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. The variable heavy chain is preferably derived from germline sequence DP-50 (GenBank LO6618) and the variable light chain is preferably derived from germline sequence L6 (GenBank X01668). The constant regions of the antibody are constant regions of human IgG1 type. Such regions can be allotypic and are described by, e.g., Johnson, G., and Wu, T. T., Nucleic Acids Res. 28 (2000) 214-218 and the databases referenced therein and are useful as long as the properties of induction of ADCC and preferably CDC according to the invention are retained.

The term “recombinant human antibody”, as used herein, is intended to include all human antibodies that are prepared, expressed, created or isolated by recombinant means, such as antibodies isolated from a host cell such as an SP2-0, NS0 or CHO cell (like CHO K1) or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, “binding” refers to antibody binding to Abeta with an affinity of about 10⁻¹³ to 10⁻⁸ M (K_{D}), preferably of about 10⁻¹³ to 10⁻⁹ M.

The “constant domains” are not involved directly in binding the antibody to an antigen but are involved in the effector functions (ADCC, complement binding, and CDC). The constant domain of an antibody according to the invention is of the IgG1 type. Human constant domains having these characteristics are described in detail by Kabat et al., Sequences of Proteins of Immunological Interest, 5th Ed. Public Health Service, National Institutes of Health, Bethesda, Md. (1991), and by Brüggemann, M., et al., J. Exp. Med. 166 (1987) 1351-1361; Love, T. W., et al., Methods Enzymol. 178 (1989) 515-527. Examples are shown in SEQ ID NOs: 5 to 8 in WO 2005/005635. Other useful and preferred constant domains are the constant domains of the antibodies obtainable from the hybridoma cell lines deposited with depositories like DSMZ or ATCC. The constant domains may provide complement binding. ADCC and optionally CDC are provided by the combination of variable and constant domains.

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop”.

The formulation of this invention may, inter alia, comprise “stabilizers”, “lyoprotectants”, “sugars”, “amino acids”, “polyols”, “antioxidants”, “preservatives”, “surfactants”, “buffers” and/or “tonicity agents”.

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland, J. L., M. F. Powell, et al. (1993). “The development of stable protein formulations: a close look at protein aggregation, deamidation, and oxidation.” Crit Rev Ther Drug Carrier Syst 10(4): 307-77, Wang, W. (1999). “Instability, stabilization, and formulation of liquid protein pharmaceuticals.” Int J Pharm 185(2): 129-88., Wang, W. (2000). “Lyophilization and development of solid protein pharmaceuticals.” Int J Pharm 203(1-2): 1-60. and Chi, E. Y., S. Krishnan, et al. (2003). “Physical stability of proteins in aqueous solution: mechanism and driving forces in nonnative protein aggregation.” Pharm Res 20(9): 1325-36. Stabilizers include but are not limited to sugars, amino acids, polyols, surfactants, antioxidants, preservatives, cyclodextrines, e.g. hydroxypropyl-β-cyclodextrine, sulfobutylethyl-β-cyclodextrin, β-Cyclodextrin, polyethylenglycols, e.g. PEG 3000, 3350, 4000, 6000, albumin, e.g. human serum albumin (HSA), bovines serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

The term “lyoprotectant” denotes pharmaceutical acceptable excipients, which protects the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. Preferred lyoprotectants can be selected from the group consisting of: sugars such as sucrose, trehalose, lactose, glucose, mannose, maltose, galactose, fructose, sorbose, and raffinose neuraminic acid and galactosamine, amino sugars such as glucosamine, N-Methylglucosamine (“Meglumine”), polyols such as mannitol, and amino acids such as arginine. Lyoprotectants are generally used in an amount of about 10 to 500 mM, preferably in an amount of about 10 to about 300 mM and more preferably in an amount of about 100 to about 300 mM.

The term “sugar” as used herein denotes a pharmaceutically acceptable carbohydrate used generally in an amount of about 10 mM to about 500 mM, preferably in an amount of about 10 to about 300 mM and more preferably in an amount of about 100 to about 300 mM. Suitable sugars comprise but are not limited to trehalose, sucrose, lactose, glucose, mannose, maltose, galactose, fructose, sorbose, raffinose, glucosamine, N-Methylglucosamine (so-called “Meglumine”), galactosamine and neuraminic acid. Preferred sugars are sucrose and trehalose and more preferably sucrose.

The term “amino acid” as used herein in the context of the pharmaceutical parenteral formulation denotes a pharmaceutical acceptable organic molecule possessing an amino moiety located at α-position to a carboxylic group. Amino acids comprise but not limited to arginine, glycine, ornithine, lysine, histidine, glutamic acid, aspartic acid, isoleucine, leucine, alanine, phenylalanine, tyrosine, tryptophan, methionine, serine, proline and combinations thereof. Amino acids are generally used in an amount of about 10 to 500 mM, preferably in an amount of about 10 to about 300 mM and more preferably in an amount of about 100 to about 300 mM.

The term “polyols” as used herein denotes pharmaceutically acceptable alcohols with more than one hydroxy group. Polyols can be used in an amount of about 10 mM to about 500 mM, preferably in an amount of about 10 to about 300 and more preferably in an amount of about 100 to about 300 mM. Suitable polyols comprise to but are not limited to mannitol, sorbitol, glycerine, dextran, glycerol, arabitol, propylene glycol, polyethylene glycol, and combinations thereof.

The term “antioxidant” denotes pharmaceutically acceptable excipients, which prevent oxidation of the active pharmaceutical ingredient. Antioxidants can be used in an amount of about 1 to about 100 mM, preferably in an amount of about 5 to about 50 mM and more preferably in an amount of about 5 to about 20 mM. Antioxidants comprise but are not limited to ascorbic acid, glutathione, cysteine, methionine, citric acid, EDTA, and combinations thereof.

The term “preservative” denotes pharmaceutically acceptable excipients, which prevent the growth of microorganism in the formulation. For example, the addition of a preservative to a multi-dose formulation protects the formulation against microbial contamination. Preservatives are generally used in an amount of about 0.001 to about 2%(w/v). Preservatives comprise but are not limited to ethanol, benzyl alcohol, phenol, m-cresol, p-chlor-m-cresol, methyl or propyl parabens, benzalkonium chloride, and combinations thereof.

The term “surfactant” as used herein denotes a pharmaceutically acceptable surfactant. In the formulation of the invention, the amount of surfactant is described a percentage expressed in weight/volume percent (w/v %). Suitable pharmaceutically acceptable surfactants comprise but are not limited to the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic)., and sodium dodecyl sulphate (SDS). Preferred polyoxyethylenesorbitan-fatty acid esters are polysorbate 20,(sold under the trademark Tween20™) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Preferred alkylphenolpolyoxyethylene ethers are sold under the tradename Triton-X. When polysorbate 20 (Tween 20™) and polysorbate 80 (Tween 80™) are used they are generally used in a concentration range of about 0.001 to about 1%, preferably of about 0.005 to about 0.1% and still preferably about 0.01% to about 0.04% w/v.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers and phosphate-buffers. Still preferred buffers comprise L-histidine or mixtures of L-histidine and L-histidine hydrochloride with pH adjustment with an acid or a base known in the art. The abovementioned histidine-buffers are generally used in an amount of about 1 mM to about 100 mM, preferably of about 5 mM to about 50 mM and still more preferably of about 10-20 mM. Independently from the buffer used, the pH can be adjusted at a value comprising about 4.0 to about 7.0 and preferably about 5.0 to about 6.0 and still preferably about 5.5 with an acid or a base known in the art, e.g., hydrochloric acid, acetic acid, phosphoric acid, sulfuric acid and citric acid, sodium hydroxide and potassium hydroxide

The term “tonicity agents” as used herein denotes pharmaceutically acceptable tonicity agents. Tonicity agents are used to modulate the tonicity of the formulation. The formulation can be hypotonic, isotonic or hypertonic. Isotonicity is generally relates to the osmotic pressure relative of a solution usually relative to that of human blood serum. The formulation according to the invention can be hypotonic, isotonic or hypertonic but will preferably be isotonic. In a concern for clarity it is once more emphasized that an isotonic formulation is liquid or liquid reconstituted from a solid form, e.g. from a lyophilized form and denotes a solution having the same tonicity as some other solution with which it is compared, such as physiologic salt solution and the blood serum. Suitable isotonicity agents comprise but are not limited to sodium chloride, potassium chloride, glycerin and any component from the group of amino acids, sugars, in particular glucose as defined herein as well as combinations thereof. Tonicity agents are used in an amount of about 5 mM to about 500 mM.

The term “liquid” as used herein in connection with the formulation according to the invention denotes a formulation which is liquid at a temperature of at least about 2 to about 8° C. under standard pressure.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. In the pharmaceutical field, the lyophilizate has usually a residual moisture of about 0.1 to 5% (w/w) and is present as a powder or a physical stable cake. The lyophilizate is characterized by a fast dissolution after addition of a reconstitution medium.

The term “reconstituted formulation” as used herein in connection with the formulation according to the invention denotes a formulation which is lyophilized and re-dissolved by addition of reconstitution medium. The reconstitution medium comprises but is not limited to water for injection (WFI), bacteriostatic water for injection (BWFI), sodium chloride solutions (e.g. 0.9% (w/v) NaCl), glucose solutions (e.g. 5% glucose), surfactant containing solutions (e.g. 0.01% polysorbate 20), a pH-buffered solution (e.g. phosphate-buffered solutions) and combinations thereof.

The term “stable formulation” as used herein in connection with the formulation according to the invention denotes a formulation, which preserves its physical and chemical integrity during manufacturing, storage and application. Various analytical techniques for evaluating protein stability are available and reviewed in Reubsaet, J. L., J. H. Beijnen, et al. (1998). “Analytical techniques used to study the degradation of proteins and peptides: chemical instability”. J Pharm Biomed Anal 17(6-7): 955-78 and Wang, W. (1999). “Instability, stabilization, and formulation of liquid protein pharmaceuticals.” Int J Pharm 185(2): 129-88. Stability can be evaluated by storage at selected climate conditions for a selected time period, by applying mechanical stress such as shaking at a selected shaking frequency for a selected time period, by irradiation with a selected light intensity for a selected period of time, or by repetitive freezing and thawing at selected temperatures.

The term “pharmaceutically acceptable” as used herein in connection with the formulation according to the invention denotes a formulation which is in compliance with the current international regulatory requirements for pharmaceuticals. A pharmaceutical acceptable formulation contains excipients which are generally recognized for the anticipated route of application and concentration range as safe. In addition, it should provide sufficient stability during manufacturing, storage and application. Especially a formulation for a parenteral route of application should fulfill the requirements isotonicity and euhydric pH in comparison to the composition of human blood.

As mentioned above, in one aspect, the invention relates to a stable pharmaceutical parenteral Abeta antibody formulation comprising:
about 1 to about 250 mg/mL Abeta antibody;
about 0.001 to about 1% of at least one surfactant;
about 1 to about 100 mM of a buffer;
optionally about 10 to about 500 mM of a stabilizer and/or about 5 to about 500 mM of a tonicity agent
at a pH of about 4.0 to about 7.0.

The Abeta antibody concentration ranges from about 1 to about 250 mg/mL, preferably from about 50 mg/mL to about 200 mg/mL and more preferably from about 150 mg/mL to about 200 mg/mL. For clarity reasons, it is emphasized that the concentrations as indicated herein relate to the concentration in a liquid or in a liquid that is accurately reconstituted from a solid form. Accordingly, the lyophilized formulations as described herein can be reconstituted from a lyophilizate in such way that the resulting reconstituted formula comprises the respective constituents in the concentrations described herein.

However, it is evident for the skilled person that the stable lyophilizates as described herein may also be reconstituted using such an amount of reconstitution medium that the resulting reconstituted formulation is either more concentrated or less concentrated. For instance, the lyophilizate of “Formulation A” as described herein in Table 2 may be reconstituted in such way that the resulting reconstituted formulation is further diluted to comprise e.g. 20 mg/mL Abeta antibody, 5.3 mM L-histidine, 66.7 mM Sucrose and 0.011% polysorbate 20; see Formulation R of Table 2.

The formulation according to the invention can be in a liquid form, a lyophilized form or in a liquid form reconstituted from a lyophilized form.

In the cases where the formulation of the invention is in a lyophilized form or in a liquid from reconstituted from a lyophilized form, it can comprise at least one lyoprotectant as stabilizer.

The formulation according to the invention can be administered by intravenous (i.v.), subcutaneous (s.c.) or any other parenteral administration means such as those known in the pharmaceutical art. The formulation according to the invention is preferably administered by subcutaneous ways.

The formulation according to the invention can be prepared by methods known in the art, such as ultrafiltration-diafiltration, dialysis, addition and mixing, lyophilisation, reconstitution, and combinations thereof. Examples of preparations of formulations according to the invention can be found hereinafter.

In a preferred embodiment, the Abeta antibody comprised in the pharmaceutical parenteral formulation of the present invention may comprise or have the variable region as defined in SEQ ID NO: 1:

(SEQ ID NO: 1)
QVELVESGGGLVQPGGSLRLSCAASGFTFSSYAMSWVRQAPGKGLEW
VSAINASGTRTYYADSVKGRFTISRDNSKNTLYLQMNSLRAEDTAVY
YCARGKGNTHKPYGYVRYFDVWGQGTLVTVSSASTKGPSVFPLAPSS
KSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYK
CKVSNKALPAPIEKTISKAKGQPREPQVYTLPPSRDELTKNQVSLTC
LVKGFYPSDIAVEWESNGQPENNYKTTPPVLDSDGSFFLYSKLTVDK
SRWQQGNVFSCSVMHEALHNHYTQKSLSLSPGK

This sequence is also depicted herein below and the CDRs, CH-regions, heavy regions as well as two N-glycosylation sites (Asn 52 and Asn 306) are indicated:

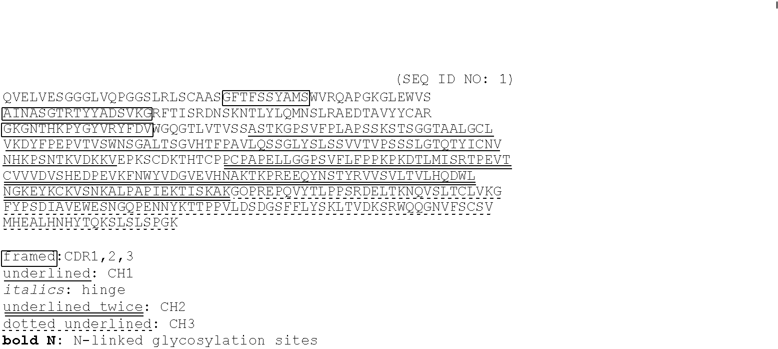

The exemplified Abeta antibody comprising SEQ ID NO: 1 as described herein may also comprise a light chain, said light chain may comprise or have the following amino acid sequence:

(SEQ ID NO: 2)
DIVLTQSPATLSLSPGERATLSCRASQSVSSSYLAWYQQKPGQAPRL
LIYGASSRATGVPARFSGSGSGTDFTLTISSLEPEDFATYYCLQIYN
MPITFGQGTKVEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC

The term “Abeta antibody A”, as used herein, relates to the exemplified Abeta antibody comprising a heavy chain as defined in SEQ ID NO: 1 and a light chain as defined in SEQ ID NO: 2.

The term “mono-glycosylated antibody(ies)”, as used herein, relates to antibody molecules comprising an N-glycosylation in one (VH)-region of an individual antibody molecule, e.g. of an immunoglobulin, e.g. an IgG, e.g. of an IgG1. For example, said “mono-glycosylated form” comprises a glycosylation on one variable region of the heavy chain e.g. at position asparagine “Asn 52” of the herein described “Abeta antibody A”. This “mono-glycosylated IgG1 -form or mono-glycosylated isoform” may also comprise, as illustrated herein, the glycosylation in the well conserved glycosylation site in the Fc-part, for example asparagine Asn 306 in the non-variable Fc-part of the herein exemplified “Abeta antibody A”.

The term “double-glycosylated antibody(ies)” in the meaning of this invention comprises the herein defined glycosylation on both variable regions of the heavy chain (VH)-region. Again, this “double glycosylated form”, comprises a glycosylation on the variable region of both heavy chains, e.g. at position asparagine Asn 52 of the herein exemplified “Abeta antibody A”. This “double-glycosylated IgG1-form or double-glycosylated isoform” may also comprise, as illustrated herein, the glycosylation in the well conserved glycosylation site in the non-variable/constant Fc-part, in particular on position 306 of the exemplified “Abeta antibody A”. Appended FIG. 1 illustrates corresponding antibody molecules.

Antibodies devoid of such a post-translational modification in the variable region, e.g. in both variable regions of the heavy chain (both (VH)-regions) are, in context of this invention considered as a “non-glycosylated form”, comprising no glycosylation in the variable region of the heavy chain. Yet, this “non-glycosylated form” may nevertheless comprise (a) glycosylation(s) in the constant region (C-region) of the antibody, for example, and most commonly at the well conserved glycosylation site of the Fc-part, in particular the asparagine (Asn) 306 in the non-variable/constant Fc-part as defined herein; see also SEQ ID NO: 1.

The pharmaceutical parenteral formulations of the invention may comprise the exemplary “Abeta antibody A” as defined herein above and as illustrated in the appended examples. Accordingly, said pharmaceutical parenteral formulations comprising Abeta antibody A may comprise mono-glycosylated Abeta antibody A or double-glycosylated Abeta antibody A or non-glycosylated Abeta antibody A or mixtures thereof as defined above.

Purification of glycosylation isoforms of recombinantly expressed Abeta antibody molecules may comprise the steps of:
(1) protein A column purification;
(2) ion exchange column purification, e.g. a cation exchange chromatography; and, optionally,
(3) size exclusion column purification.

The purification protocol may comprise further steps, like further concentration steps, e.g. diafiltration or analytical steps, e.g. involving analytical columns. It is also envisaged and feasible that particular certain steps are repeated (e.g. two ion exchange chromatography steps may be carried out) or that certain steps (e.g. size exclusion chromatography) may be omitted.

Protein A is a group specific ligand which binds to the Fc region of most IgG1 isotypes. It is synthesized by some strains of Staphylococcus aureus and can be isolated therefrom and coupled to chromatographic beads. Several types of gel preparations are available commercially. An example for a protein A column which may be used is a MabSelect (Trademark) column. Ideally the column is equilibrated with 25 mM Tris/HCl, 25 mM NaCl, 5 mM EDTA, the cell culture supernatant is loaded onto the column, the column is washed with 1 M Tris/HCl pH 7.2 and the antibody is eluted at pH 3.2 using 100 mM acetic acid.

Cation-exchange chromatography exploits interactions between positively charged groups in a stationary phase and the sample which is in the mobile phase. When a weak cation exchanger (e.g. CM Toyopearl 650®) is used, the following chromatographic steps are performed: After preequilibration with 100 mM acetic acid pH 4, loading of Protein A eluate and washing with 100 mM acetic acid pH 4 the antibody is eluted and fractionated by applying steps of 250 mM sodium acetate (pH 7.8-8.5) and 500 mM sodium acetate (pH 7.8-8.5). With the first step a mixture of double-glycosylated isoform fraction and mono-glycosylated isoform fraction are normally eluted, using the second step the non-glycosylated isoform fraction is normally eluted.

From a strong cation exchanger (e.g. SP Toyopearl 650) the antibody can be eluted by salt steps: After equilibration of the column with 50 mM acetic acid pH 5.0, loading the Protein A eluate with pH 4 the first elution step using 50 mM acetic acid and 210 mM sodium chloride is performed. Then a second elution step of 50 mM acetic acid and 350 mM sodium chloride is applied. By the first salt step a mixture of the double-glycosylated isoform fraction and mono-glycosylated isoform fraction are normally eluted, by the second salt step the non-glycosylated isoform is normally eluted.

In addition the antibody may also be eluted from a strong cation exchanger column (e.g. SP-Sepharose®) by a salt gradient: After preequilibration, loading and washing the column at pH 4.5 a salt gradient is applied from 50 mM MES pH 5.8 to 50 mM MES/1 M sodium chloride pH 5.8. Here the double-glycosylated isoform, mono-glycosylated isoform and non-glycosylated isoform fractions are normally eluted separately. In the following double-glycosylated isoform fraction and mono-glycosylated isoform fraction may be pooled to result in the product pool and/or a desired antibody mixture.

Further purification of the mixture of double- and mono-glycosylated antibody molecules, e.g. immunoglobulins, may be performed by size exclusion chromatography. An example of a useful column is a Superdex 200® column. Examples of running buffers include histidine/sodium chloride, e.g. 10 mM histidine/125 mM sodium chloride/pH 6, and phosphate buffered saline (PBS).

Anion exchange chromatography in the flow through mode followed by a concentration/diafiltration is an alternative purification step. Q Sepharose® is an example for a resin for the anion exchange step. For example, the eluate from the SP chromatography may be threefold diluted with 37,5 mM Tris/HCl pH 7.9 and passed over a Q-Sepharose column pre-equilibrated with 25 mM Tris/83 mM sodium acetate. The flow through is collected, adjusted to pH 5.5 and concentrated by ultrafiltration using e.g. a Hydrosart 30 kD® membrane. In the following the concentrate may be diafiltrated against for example 10 volumes of 20 mM histidine/HCl pH 5.5.

As defined above, antibody isoforms may also comprise (a) further glycosylation(s) in the constant/non-variable part of the antibody molecules, e.g. in the Fc-part of an IgG, e.g. in the Fc-part in an IgG1. Said glycosylation in the Fc-part relates to a well conserved glycosylation, being characterized in located on position Asn306 of the heavy chain, e.g., in accordance with the herein defined SEQ ID NO: 1.

The IgG-Fc region of the antibodies comprised in the formulations of this invention may be a homodimer comprised of inter-chain disulphide bonded hinge regions, glycosylated CH2 domains, bearing N-linked oligosaccharide at asparagine 306 (Asn-306) of the CH2 and non-covalently paired CH3 domains. The oligosaccharide of the glycosylation at Asn-306 is of the complex biantennary type and may comprise a core heptasaccharide structure with variable addition of outer arm sugars.

The oligosaccharide influences or determines Fc structure and function (Jefferis (1998) Immunol Rev. 163, 50-76). Effector functions, numbering particular specific IgG-Fc/effector ligand interactions have been discussed (Jefferis (2002) Immunol Lett. 82(1-2), 57-65 and Krapp (2003) J Mol Biol. 325(5), 979-89). This conserved Fc-position Asn-306 corresponds to “Asn-297” in the Kabat-system (Kabat (1991) Sequences of Proteins of Immunological Interest, 5th Ed., Public Health Service, National Institutes of Health, Bethesda Md.)

In a certain embodiment, the formulation of the invention is a liquid or lyophilized formulation comprising:
about 1 to about 200 mg/mL Abeta antibody,
0.04% Tween 20 w/v,
20 mM L-histidine,
250 mM Sucrose,
at pH 5.5.

In another embodiment, the formulation according to the invention also comprises a lyophilized formulation comprising:
75 mg/mL Abeta antibody,
0.04% Tween 20 w/v,
20 mM L-histidine,
250 mM Sucrose,
at pH 5.5.
   or
75 mg/mL Abeta antibody,
0.02% Tween 20 w/v,
20 mM L-histidine,
250 mM Sucrose,
at pH 5.5.

In yet another embodiment, the formulation according to the invention also comprises a liquid formulation comprising:
37.5 mg/mL Abeta antibody,
0.02% Tween 20 w/v,
10 mM L-histidine,
125 mM Sucrose,
at pH 5.5.
   or
37.5 mg/mL Abeta antibody,
0.01% Tween 20 w/v,
10 mM L-histidine,
125 mM Sucrose,
at pH 5.5.

In still another embodiment, the formulation according to the invention also comprises a lyophilized formulation comprising:
15 mg/mL Abeta antibody,
0.04% Tween 20 w/v,
20 mM L-histidine,
250 mM Sucrose,
at pH 5.5.

In still another embodiment, the formulation according to the invention also comprises a lyophilized formulation comprising:
20 mg/mL Abeta antibody,
0.011% Tween 20 w/v,
5.3 mM L-histidine,
66.7 mM Sucrose,
at pH 5.5.

In still another embodiment, the formulation according to the invention also comprises a liquid formulation comprising:
7.5 mg/mL Abeta antibody,
0.04% Tween 20 w/v,
20 mM L-histidine,
250 mM Sucrose,
at pH 5.5;
   or
7.5 mg/mL Abeta antibody,
0.02% Tween 20 w/v,
10 mM L-histidine,
125 mM Sucrose,
at pH 5.5.

In a further embodiment, the formulation according to the invention also comprises a lyophilized formulation comprising:
75 mg/mL Abeta antibody,
0.04% Tween 20 w/v,
20 mM L-histidine,
250 mM Trehalose,
at pH 5.5.
   or
75 mg/mL Abeta antibody,
0.02% Tween 20 w/v,
20 mM L-histidine,
250 mM Trehalose,
at pH 5.5.

In still another embodiment, the formulation according to the invention also comprises a liquid formulation comprising:
37.5 mg/mL Abeta antibody,
0.02% Tween 20 w/v,
10 mM L-histidine,
125 mM Trehalose,
at pH 5.5.
   or
37.5 mg/mL Abeta antibody,
0.01% Tween 20 w/v,
10 mM L-histidine,
125 mM Trehalose,
at pH 5.5.

In still another embodiment, the formulation according to the invention also comprises a liquid formulation comprising:
75 mg/mL Abeta antibody,
0.02% Tween 20 w/v,
20 mM L-histidine,
250 mM Trehalose,
at pH 5.5.
   or
75 mg/mL Abeta antibody,
0.02% Tween 20 w/v,
20 mM L-histidine,
250 mM Mannitol,
at pH 5.5.
   or
75 mg/mL Abeta antibody,
0.02% Tween 20 w/v,
20 mM L-histidine,
140 mM Sodium Chloride,
at pH 5.5.
   or
150 mg/mL Abeta antibody,
0.02% Tween 20 w/v,
20 mM L-histidine,
250 mM Trehalose,
at pH 5.5.
   or
150 mg/mL Abeta antibody,
0.02% Tween 20 w/v,
20 mM L-histidine,
250 mM Mannitol,
at pH 5.5.
   or
150 mg/mL Abeta antibody,
0.02% Tween 20 w/v,
20 mM L-histidine,
140 mM Sodium Chloride,
at pH 5.5.
   or
10 mg/mL Abeta antibody,
0.01% Tween 20 w/v,
20 mM L-histidine,
140 mM Sodium chloride,
at pH 5.5

In a preferred embodiment, the formulation according to the invention also comprises a liquid formulation comprising:
10 mg/mL Abeta antibody,
0.01% Tween 20 w/v,
20 mM L-histidine,
140 mM Sodium chloride,
at pH 5.5

In another preferred embodiment, the formulation according to the invention also comprises a lyophilized formulation comprising:
75 mg/mL Abeta antibody,
0.04% Tween 20 w/v,
20 mM L-histidine,
250 mM Sucrose,
at pH 5.5

In another preferred embodiment, the formulation according to the invention also comprises a lyophilized formulation comprising:
20 mg/mL Abeta antibody,
0.011% Tween 20 w/v,
5.3 mM L-histidine,
66.7 mM Sucrose
at pH 5.5

### FIGURE LEGENDS

FIG. 1 Scheme of double-, mono- and non-glycosylated antibody molecules (immunoglobulins).

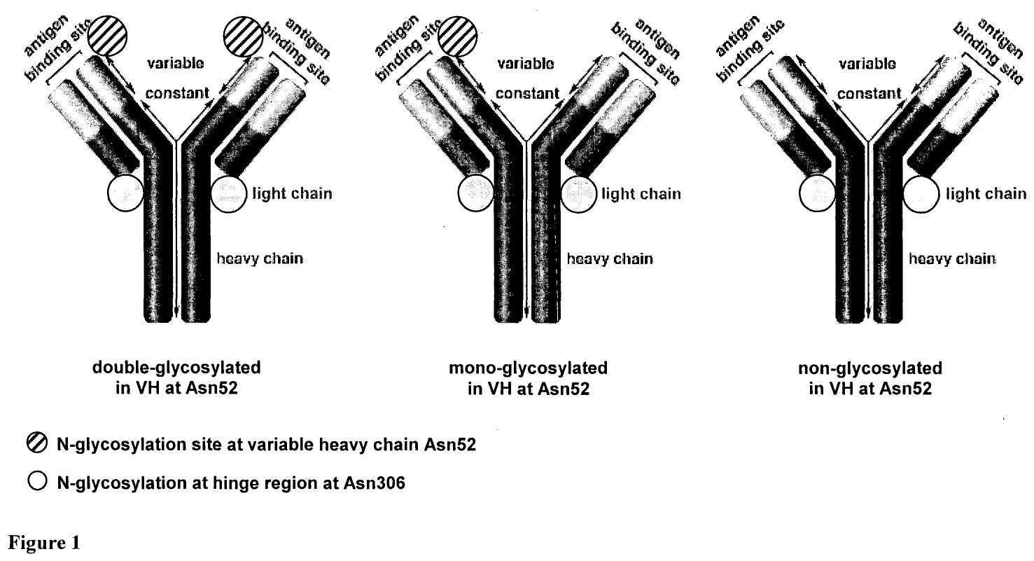

FIG. 2 Content of monomer as determined by size-exclusion chromatography of Abeta antibody A formulations after start and incubation at 5° C., 25° C./60% rh and 40° C./75% rh for up to 6 months. Antibody preparations are freeze-dried and reconstituted to nominal concentration of 75 mg/mL.

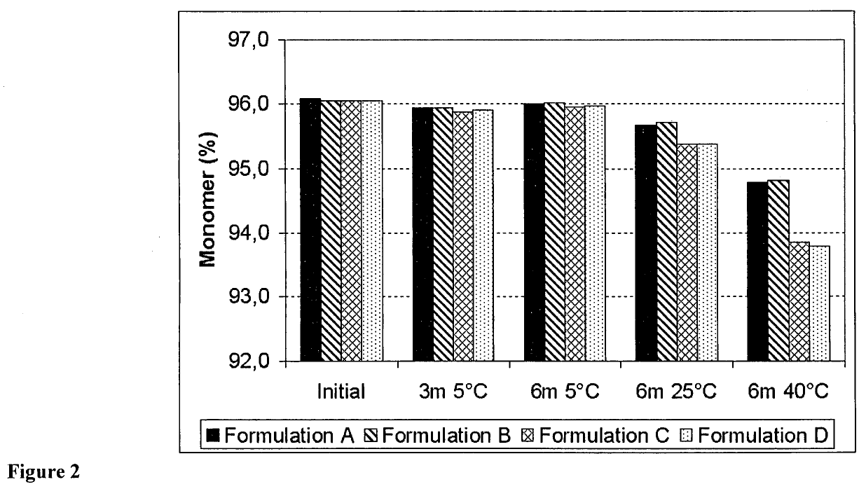

FIG. 3 Content of monomer as determined by size-exclusion chromatography of Abeta antibody A formulations after start and incubation at 5° C., 25° C./60% rh and 40° C./75% rh for 3 months. Antibody preparations K, L and N are formulated at 75 mg/mL, whereas preparations O, P and Q are formulated at 150 mg/mL.

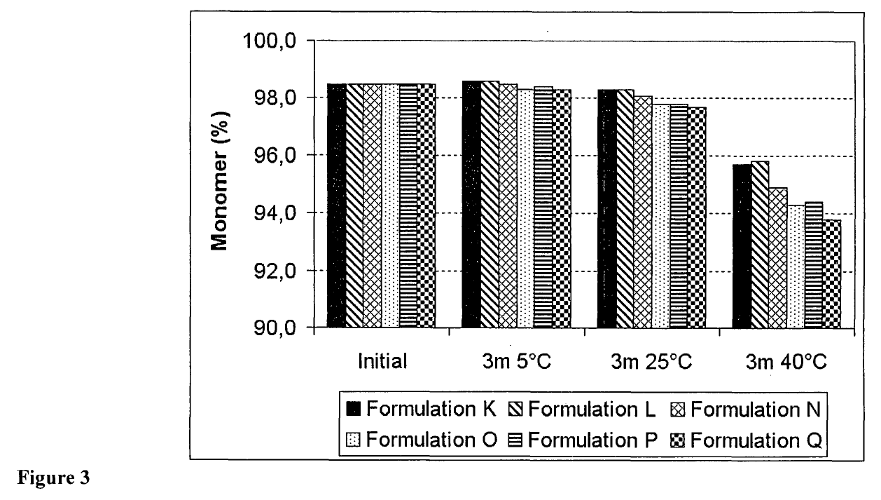

### EXAMPLES

Liquid and lyophilized drug product formulations for subcutaneous administration according to the invention were developed as follows:

Preparation of Liquid Formulations

Abeta antibody comprising a heavy chain as defined in SEQ ID NO: 1 and a light chain as defined in SEQ ID NO: 2 (“Abeta antibody A” in the context of the present invention) was prepared and obtained as described in WO 03/070760 and was concentrated by ultrafiltration to a concentration of approx. 40 to about 200 mg/mL in a 20 mM histidine buffer at a pH of approx. 5.5. The concentrated solution was then diluted with the formulation buffer (containing sugar (respectively salt or polyol), surfactant and buffer at a pH of approx. pH 5.5) resulting the anticipated antibody concentration of approx. 7.5 mg/mL, 37.5 mg/mL, 75 mg/mL or 150 mg/mL formulated in the final bulk composition (e.g. 10 mM L-histidine, 125 mM sucrose, 0.02% Tween 20, at pH 5.5).

Alternatively, Abeta antibody A was buffer-exchanged against a diafiltration buffer containing the anticipated buffer and sugar composition and concentrated to an antibody concentration equal or higher than the final concentration of approx. 37.5 mg/mL. The surfactant was added after completion of the ultrafiltration operation as a 100 to 200-fold stock solution to the antibody solution. The concentrated antibody solution was adjusted with a formulation buffer containing the identical excipient composition to the final Abeta antibody A concentration of approx. 37.5 mg/mL.

All formulations were sterile-filtered through 0.22 ⱼim low protein binding filters and aseptically filled under nitrogen atmosphere into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions for different intervals of time and stressed by shaking (1 week at a shaking frequency of 200 min⁻¹ at 5° C.) and freeze-thaw stress methods. The samples were analyzed before and after applying the stress tests by the analytical methods 1) UV spectrophotometry, 2) Size Exclusion Chromatography (SEC) and 3) nephelometry to determine the turbidity of the solution.

Preparation of Lyophilized Formulations and Liquid Formulations Reconstituted from such Lyophilized Formulations

Solutions of approx. 37.5 mg/ml “Abeta antibody A” were prepared as described above for liquid formulations. Any lyophilization method known in the art is intended to be within the scope of the invention. For example, the lyophilization process used for this study included the cooling of the formulation from room temperature to approx 5° C. (pre-cooling) and a freezing step to −40° C. at a plate cooling rate of approx. 1 ° C./min, followed by a holding step at −40° C. for about 2 hours. The first drying step was performed at a plate temperature of approx. −25° C. and a chamber pressure of approx. 80 μbar for about 62 hours. Subsequently, the second drying step started with a temperature ramp of 0.2° C./min from −25° C. to 25° C., followed by a holding step at 25° C. for at least 5 hours at a chamber pressure of approx. 80 μbar (the applied drying schedule is presented in Table 1.)

Lyophilization was carried out in an Usifroid SMH-90 LN2 freeze-dryer (Usifroid, Maurepas, France). All lyophilized cakes in this study had a residual water content of about 0.1 to 1.0% as determined by Karl-Fischer method. The freeze-dried samples were incubated at different temperatures for different intervals of time.

The lyophilized formulations were reconstituted to a final volume of 1.2 mL with water for injection (WFI) yielding an isotonic formulation with an antibody concentration of approx. 75 mg/mL and a viscosity of less than 3 mPa·s. The reconstitution time of the freeze-dried cakes was about 2 to 4 min. Analysis of the reconstituted samples was either performed immediately after reconstitution, or after a 24 hour incubation period of the reconstituted liquid sample at 25° C.

The samples were analyzed by 1) UV spectrophotometry, 2) determination of the reconstitution time, 3) Size Exclusion Chromatography (SEC) and 4) method of nephelometry to determine the turbidity of the solution.

Size Exclusion Chromatography (SEC) was used to detect soluble high molecular weight species (aggregates) and low molecular weight hydrolysis products (LMW) in the formulations. The method was performed on a Merck Hitachi 7000 HPLC instrument equipped with a Tosohaas TSK G3000 SWXL column. Intact monomer, aggregates and hydrolysis products are separated by an isocratic elution profile, using 0.2M K₂HPO₄/0.25M KCL, pH 7.0 as mobile phase, and were detected at a wavelength of 280 nm.

UV spectroscopy, used for determination of protein content, was performed on a Varian Cary Bio UV spectrophotometer at 280 nm. Neat protein samples were diluted to approx. 0.5 mg/mL with 20 mM L-histidine, pH 5.5. The protein concentration was calculated according equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The protein concentration was measured with a precision of ±10%. The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient c.

Clarity and the degree of opalescence were measured as Formazin Turbidity Units (FTU) by the method of nephelometry. The neat sample was transferred into a 11 mm diameter clear-glass tube and placed into a HACH 2100AN turbidimeter.

TABLE 1
Freeze-drying Cycle type I

| | Shelf | | | Vacuum Set |
|---|---|---|---|---|
| | temperature | Ramp Rate | Hold time | point |
| Step | (° C.) | (° C./min) | (min) | (μbar) |
| Pre-cooling | 5° C. | 0.0 | 60 | — |
| Freezing | −40° C. | 1.0 | 150 | — |
| Primary Drying | −25° C. | 0.5 | 3700 | 80 |
| Secondary | +25° C. | 0.2 | 300 | 80 |
| Drying | | | | |

TABLE 2
Compositions of “Abeta antibody A” drug product
formulations according to the invention

| | | | Composition | | |
|---|---|---|---|---|---|
| Formulation | | | (Stability data in Table) | | |
| Lyophilized Formulations | | | | | |
| Formulation A | | | 75 mg/mL Abeta antibody A, | | |
| | | | 20 mM L-histidine, | | |
| | | | 250 mM Sucrose, | | |
| | | | 0.04% polysorbate 20, | | |
| | | | at pH 5.5 | | |
| | Protein | | | | Turbidity |
| | conc. after | Size Exclusion - HPLC | | | after |
| | reconst. (*) | HMW | Monomer | LMW | reconst. |
| Timepoint | (mg/mL) | (%) | (%) | (%) | (FTU) |
| Initial | 72.8 | 1.9 | 96.1 | 2.0 | 5.4 |
| 24 h at | 74.8 | 1.9 | 96.0 | 2.1 | 5.3 |
| 25° C. after | | | | | |
| reconst. | | | | | |
| 1 month | 74.5 | 1.7 | 95.8 | 2.5 | 5.4 |
| at 2-8° C. | | | | | |
| 3 months | 74.2 | 2.0 | 95.9 | 2.1 | 5.6 |
| at 2-8° C. | | | | | |
| 6 months | n.d. | 2.0 | 96.0 | 2.0 | n.d. |
| at 2-8° C. | | | | | |
| 6 months at | n.d | 2.3 | 95.7 | 2.0 | n.d |
| 25° C./60% rh | | | | | |
| 6 months at | n.d. | 3.2 | 94.8 | 2.0 | n.d. |
| 40° C./75% rh | | | | | |
| Formulation B | | | 75 mg/mL Abeta antibody A, | | |
| | | | 20 mM L-histidine, | | |
| | | | 250 mM Sucrose, | | |
| | | | 0.02% polysorbate 20, | | |
| | | | at pH 5.5 | | |
| | Protein | | | | Turbidity |
| | conc. after | Size Exclusion - HPLC | | | after |
| | reconst. (*) | HMW | Monomer | LMW | reconst. |
| Timepoint | (mg/mL) | (%) | (%) | (%) | (FTU) |
| Initial | 74.9 | 1.9 | 96.1 | 2.0 | 5.3 |
| 24 h at | 73.8 | 1.9 | 96.1 | 2.0 | 5.2 |
| 25° C. after | | | | | |
| reconst. | | | | | |
| 1 month | 74.3 | 1.7 | 95.9 | 2.4 | 5.4 |
| at 2-8° C. | | | | | |
| 3 months | 73.9 | 2.0 | 95.9 | 2.1 | 6.0 |
| at 2-8° C. | | | | | |
| 6 months | n.d. | 2.0 | 96.0 | 2.0 | n.d. |
| at 2-8° C. | | | | | |
| 6 months at | n.d | 2.3 | 95.7 | 2.0 | n.d |
| 25° C./60% rh | | | | | |
| 6 months at | n.d. | 3.2 | 94.8 | 2.0 | n.d. |
| 40° C./75% rh | | | | | |
| Formulation C | | | 75 mg/mL Abeta antibody A, | | |
| | | | 20 mM L-histidine, | | |
| | | | 250 mM Trehalose, | | |
| | | | 0.04% polysorbate 20, | | |
| | | | at pH 5.5 | | |
| | Protein | | | | Turbidity |
| | conc. after | Size Exclusion - HPLC | | | after |
| | reconst. (*) | HMW | Monomer | LMW | reconst. |
| Timepoint | (mg/mL) | (%) | (%) | (%) | (FTU) |
| Initial | 74.4 | 2.0 | 96.1 | 2.0 | 5.3 |
| 24 h at | 73.6 | 2.0 | 96.0 | 2.1 | 5.1 |
| 25° C. after | | | | | |
| reconst. | | | | | |
| 1 month | 72.7 | 1.7 | 95.7-95.9 | 2.4 | 5.3 |
| at 2-8° C. | | | | | |
| 3 months | 72.5 | 2.0 | 95.9 | 2.1 | 5.2 |
| at 2-8° C. | | | | | |
| 6 months | n.d. | 2.0 | 96.0 | 2.0 | n.d. |
| at 2-8° C. | | | | | |
| 6 months at | n.d | 2.6 | 95.4 | 2.0 | n.d |
| 25° C./60% rh | | | | | |
| 6 months at | n.d. | 4.2 | 93.8 | 2.0 | n.d. |
| 40° C./75% rh | | | | | |
| Formulation D | | | 75 mg/mL Abeta antibody A, | | |
| | | | 20 mM L-histidine, | | |
| | | | 250 mM Trehalose, | | |
| | | | 0.02% polysorbate 20, | | |
| | | | at pH 5.5 | | |
| | Protein | | | | Turbidity |
| | conc. after | Size Exclusion - HPLC | | | after |
| | reconst. (*) | HMW | Monomer | LMW | reconst. |
| Timepoint | (mg/mL) | (%) | (%) | (%) | (FTU) |
| Initial | 73.6 | 2.0 | 96.1 | 2.0 | 5.2 |
| 24 h at | 72.8 | 2.0 | 96.0 | 2.0 | 5.6 |
| 25° C. after | | | | | |
| reconst. | | | | | |
| 1 month | 72.9 | 1.8 | 95.8 | 2.4 | 5.1 |
| at 2-8° C. | | | | | |
| 3 months | 73.4 | 2.0 | 95.9 | 2.1 | 5.5 |
| at 2-8° C. | | | | | |
| 6 months | n.d. | 2.0 | 96.0 | 2.0 | n.d. |
| at 2-8° C. | | | | | |
| 6 months at | n.d | 2.6 | 95.4 | 2.0 | n.d. |
| 25° C./60% rh | | | | | |
| 6 months at | n.d. | 4.2 | 93.8 | 2.0 | n.d. |
| 40° C./75% rh | | | | | |
| Formulation E | | | 15 mg/mL Abeta antibody A, | | |
| | | | 20 mM L-histidine, | | |
| | | | 250 mM Sucrose, | | |
| | | | 0.04% polysorbate 20, | | |
| | | | at pH 5.5 | | |
| Liquid Formulations | | | | | |
| Formulation F | | | 37.5 mg/mL Abeta antibody A, | | |
| Storage at 2-8° C. | | | 10 mM L-histidine, | | |
| | | | 125 mM Sucrose, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 36.7 | 1.8 | 96.2 | 2.0 | 3.5 |
| 1 week | 36.8 | 1.8 | 96.2 | 2.0 | 3.6 |
| shaking | | | | | |
| 3 months | 37.8 | 1.8 | 96.1 | 2.1 | 3.4 |
| Formulation G | | | 37.5 mg/mL Abeta antibody A, | | |
| Storage at 2-8° C. | | | 10 mM L-histidine, | | |
| | | | 125 mM Sucrose, | | |
| | | | 0.01% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 36.8 | 1.8 | 96.2 | 2.0 | 3.3 |
| 1 week | 36.8 | 1.8 | 96.3 | 1.9 | 3.6 |
| shaking | | | | | |
| 3 months | 37.8 | 1.8 | 96.1 | 2.1 | 3.9 |
| Formulation H | | | 37.5 mg/mL Abeta antibody A, | | |
| Storage at 2-8° C. | | | 10 mM L-histidine, | | |
| | | | 125 mM Trehalose, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 36.6 | 1.8 | 96.2 | 2.0 | 3.6 |
| 1 week | 36.6 | 1.8 | 96.2 | 2.0 | 3.4 |
| shaking. | | | | | |
| 3 months | 37.7 | 1.8 | 96.1 | 2.1 | 4.2 |
| Formulation I | | | 37.5 mg/mL Abeta antibody A, | | |
| Storage at 2-8° C. | | | 10 mM L-histidine, | | |
| | | | 125 mM Trehalose, | | |
| | | | 0.01% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 36.6 | 1.8 | 96.2 | 2.0 | 3.5 |
| 1 week | 36.4 | 1.8 | 96.2 | 2.0 | 3.5 |
| shaking. | | | | | |
| 3 months | 37.8 | 1.8 | 96.1 | 2.1 | 3.7 |
| Formulation J | | | 7.5 mg/mL Abeta antibody A, | | |
| | | | 10 mM L-histidine, | | |
| | | | 125 mM Sucrose, | | |
| | | | 0.02% polysorbate 20, | | |
| | | | at pH 5.5 | | |
| Formulation K | | | 75 mg/mL Abeta antibody A, | | |
| | | | 20 mM L-histidine, | | |
| | | | 250 mM Trehalose, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 75.3 | 0.9 | 98.5 | 0.6 | 5.0 |
| 1 week | 77.0 | 0.8 | 98.6 | 0.6 | 4.9 |
| shaking, | | | | | |
| at 2-8° C. | | | | | |
| 3 months | 70.5 | 0.8 | 98.6 | 0.6 | 5.2 |
| at 2-8° C. | | | | | |
| 3 months at | 72.0 | 0.9 | 98.3 | 0.8 | 8.1 |
| 25° C./60% rh | | | | | |
| 3 months at | 69.1 | 1.5 | 95.7 | 2.9 | 6.9 |
| 40° C./75% rh | | | | | |
| Formulation L | | | 75 mg/mL Abeta antibody A, | | |
| | | | 20 mM L-histidine, | | |
| | | | 250 mM Mannitol, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 76.6 | 0.9 | 98.5 | 0.6 | 5.7 |
| 1 week | 77.4 | 0.8 | 98.6 | 0.6 | 5.5 |
| shaking, | | | | | |
| at 2-8° C. | | | | | |
| 3 months | 81.1 | 0.8 | 98.6 | 0.6 | 5.7 |
| at 2-8° C. | | | | | |
| 3 months at | 72.0 | 0.9 | 98.3 | 0.8 | 8.4 |
| 25° C./60% rh | | | | | |
| 3 months at | 72.9 | 1.4 | 95.8 | 2.8 | 8.6 |
| 40° C./75% rh | | | | | |
| Formulation M | | | 10 mg/mL Abeta antibody A, | | |
| Storage at 2-8° C. | | | 20 mM L-histidine, | | |
| | | | 140 mM Sodium chloride, | | |
| | | | 0.01% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 9.7 | 0.7 | 98.1 | 1.2 | 3.7 |
| 1 week | 9.7 | 0.7 | 98.0 | 1.3 | 3.8 |
| shaking | | | | | |
| 3 months | 9.6 | 0.7 | 98.0 | 1.3 | 3.7 |
| Formulation N | | | 75 mg/mL Abeta antibody A, | | |
| | | | 20 mM L-histidine, | | |
| | | | 140 mM Sodium Chloride, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 73.9 | 1.0 | 98.5 | 0.6 | 17.5 |
| 1 week | 80.0 | 0.9 | 98.5 | 0.6 | 18.7 |
| shaking. | | | | | |
| at 2-8° C. | | | | | |
| 3 months | 74.5 | 1.0 | 98.5 | 0.6 | 18.6 |
| at 2-8° C. | | | | | |
| 3 months at | 72.1 | 1.1 | 98.1 | 0.8 | 19.4 |
| 25° C./60% rh | | | | | |
| 3 months at | 70.4 | 2.1 | 94.9 | 3.0 | n.d. |
| 40° C./75% rh | | | | | |
| Formulation O | | | 150 mg/mL Abeta antibody A, | | |
| | | | 20 mM L-histidine, | | |
| | | | 250 mM Trehalose, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 143.7 | 1.0 | 98.5 | 0.6 | 5.7 |
| 1 week | 151.9 | 1.0 | 98.5 | 0.6 | 5.0 |
| shaking. | | | | | |
| at 2-8° C. | | | | | |
| 3 months | 138.1 | 1.1 | 98.3 | 0.6 | 5.5 |
| at 2-8° C. | | | | | |
| 3 months at | 134.5 | 1.5 | 97.8 | 0.8 | 7.3 |
| 25° C./60% rh | | | | | |
| 3 months at | 141.7 | 3.0 | 94.3 | 2.8 | 6.2 |
| 40° C./75% rh | | | | | |
| Formulation P | | | 150 mg/mL Abeta antibody A, | | |
| | | | 20 mM L-histidine, | | |
| | | | 250 mM Mannitol, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 146.4 | 1.0 | 98.5 | 0.6 | 5.8 |
| 1 week | 153.4 | 1.0 | 98.5 | 0.6 | 5.3 |
| shaking. | | | | | |
| at 2-8° C. | | | | | |
| 3 months | 141.1 | 1.1 | 98.4 | 0.6 | 5.9 |
| at 2-8° C. | | | | | |
| 3 months at | 146.7 | 1.5 | 97.8 | 0.8 | 7.1 |
| 25° C./60% rh | | | | | |
| 3 months at | 138.1 | 2.8 | 94.4 | 2.8 | 7.1 |
| 40° C./75% rh | | | | | |
| Formulation Q | | | 150 mg/mL Abeta antibody A, | | |
| | | | 20 mM L-histidine, | | |
| | | | 140 mM Sodium Chloride, | | |
| | | | 0.02% polysorbate 20, | | |
| | | | at pH 5.5 | | |
| | Protein | Size Exclusion - HPLC | | | |
| | conc. | HMW | Monomer | LMW | Turbidity |
| Timepoint | (mg/mL) | (%) | (%) | (%) | (FTU) |
| Initial | 150.8 | 1.0 | 98.5 | 0.6 | 18.0 |
| 1 week | 158.3 | 1.0 | 98.5 | 0.6 | 19.0 |
| shaking. | | | | | |
| at 2-8° C. | | | | | |
| 3 months | 136.0 | 1.1 | 98.3 | 0.6 | 17.5 |
| at 2-8° C. | | | | | |
| 3 months at | 148.5 | 1.6 | 97.7 | 0.8 | 19.0 |
| 25° C./60% rh | | | | | |
| 3 months at | 144.4 | 3.4 | 93.8 | 2.8 | 19.6 |
| 40° C./75% rh | | | | | |
| lyophilized Formulation | | | | | |
| Formulation R | | | 20 mg/mL Abeta antibody A, | | |
| | | | 5.3 mM L-histidine, | | |
| | | | 66.7 mM Sucrose, | | |
| | | | 0.011% polysorbate 20, | | |
| | | | at pH 5.5 | | |
| | Protein | | | | Turbidity |
| | conc. after | Size Exclusion - HPLC | | | after |
| | reconst. (*) | HMW | Monomer | LMW | reconst. |
| Timepoint | (mg/mL) | (%) | (%) | (%) | (FTU) |
| Initial | 19.4 | 0.8 | 99.1 | 0.1 | 1.4 |
| 1 month | 19.6 | 0.8 | 99.1 | 0.1 | 1.5 |
| at 2-8° C. | | | | | |
| 3 months | 19.4 | 0.8 | 99.1 | 0.1 | 1.5 |
| at 2-8° C. | | | | | |
| 3 months at | 19.5 | 1.0 | 98.9 | 0.1 | 1.6 |
| 25° C./60% rh | | | | | |
| 3 months at | 19.5 | 1.7 | 98.2 | 0.1 | 1.6 |
| 40° C./75% rh | | | | | |

(*) taking into account the analytical precision and slight variability of reconstitution.

1. A stable pharmaceutical parenteral Abeta antibody formulation comprising:
about 1 to about 250 mg/mL Abeta antibody;
about 0.001 to about 1% of at least one surfactant;
about 1 to about 100 mM of a buffer;
optionally about 10 to about 500 mM of a stabilizer and/or about 5 to about 500 mM of a tonicity agent;
at a pH of about 4.0 to about 7.0.

2. The formulation according to claim 1 wherein it is a liquid formulation.

3. The formulation according to claim 1 wherein it is a lyophilized formulation.

4. The formulation according to claim 1 wherein it is a liquid formulation reconstituted from a lyophilized formulation.

5. The formulation according to claim 1, wherein the Abeta antibody concentration is of about 1 to about 200 mg/mL.

6. The formulation according to claim 5 wherein the Abeta antibody concentration is of about 50 mg/mL to about 200 mg/mL.

7. The formulation according to claim 6 wherein the Abeta antibody concentration is of about 150 mg/mL to about 200 mg/mL.

8. The formulation according to claim 1, wherein the stabilizer is present in the formulation in an amount of about 10 to about 300 mM.

9. The formulation according to claims 1, wherein the stabilizer is present in the formulation in an amount of about 100 to about 300 mM

10. The formulation according to claim 1, wherein the stabilizer is selected from the group consisting of sugars, amino acids, polyols, surfactants, antioxidants, preservatives, cyclodextrines, in particular hydroxypropyl-β-cyclodextrine, sulfobutylethyl-β-cyclodextrin and β-cyclodextrin, polyethylenglycols, in particular PEG 3000, 3350, 4000 and 6000, albumin, human serum albumin (HSA), bovines serum albumin (BSA), salts in particular sodium chloride, magnesium chloride, calcium chloride and chelators, in particular EDTA.

11. The formulation according to claim 1, wherein the stabilizer is a lyoprotectant.

12. The formulation according to claim 11, wherein the lyoprotectant is selected from the group consisting of sugars, amino acids, polyols and sugar alcohols.

13. The formulation according to claim 12, wherein the lyoprotectant is selected from the group consisting of trehalose, sucrose, mannitol, lactose, glucose, mannose, maltose, galactose, fructose, sorbose, raffinose, glucosamine, N-Methylglucosamine (“Meglumine”), galactosamine, neuraminic acid and arginine.

14. The formulation according to claim 1, wherein the surfactant is present in the formulation in an amount of about 0.005 to about 0.1% w/v.

15. The formulation according to claim 14, wherein the surfactant is present in the formulation in an amount of about 0.01% to about 0.04%w/v.

16. The formulation according to claim 1, wherein the surfactant is selected from the group consisting of polyoxyethylensorbitan fatty acid esters, polyoxyethylene alkyl ethers, alkylphenylpolyoxyethylene ethers, polyoxyethylene-polyoxypropylene copolymer and sodium dodecyl sulphate

17. The formulation according to claim 16, wherein the surfactant is selected from the group of polyoxyethylene sorbitan monolaureate and polyoxyethylene sorbitan monooleate, poloxamer 124, poloxamer 188, poloxamer 237, poloxamer 338 and poloxamer 407, polyoxyethylene (23) lauryl ether, polyoxyethylene (20) cetyl ether, polyoxyethylene (10) oleyl ether and polyoxyethylene (20) oleyl ether, and octyl phenol ethoxylate (7.5), octyl phenol ethoxylate (9.5), and octyl phenol ethoxylate (102).

18. The formulation according to claim 17, wherein the surfactant is selected from the group containing polyoxyethylene sorbitan monolaureate and polyoxyethylene sorbitan monooleate

19. The formulation according to claim 1, wherein the buffer is present in the formulation in an amount of about 1 mM to about 100 mM.

20. The formulation according to claim 19, wherein the buffer is present in the formulation in an amount of about 5 mM to about 50 mM.

21. The formulation according to claim 20, wherein the buffer is present in the formulation in an amount of about 10 to about 20 mM.

22. The formulation according to claim 1, wherein the buffer is selected from the group consisting of histidine-buffers, citrate-buffers, succinate-buffers, acetate-buffers and phosphate-buffers.

23. The formulation according to claim 22 wherein the buffer comprises L-histidine or mixtures of L-histidine with L-histidine hydrochloride.

24. The formulation according to claim 1, wherein the pH is about 4.0 to about 7.0.

25. The formulation according to claim 24, wherein the pH is about 5.0 to about 6.0.

26. The formulation according to claim 25, wherein the pH is about 5.5.

27. The formulation according to claim 1, which comprises one or more tonicity agents.

28. The formulation according to claim 27, wherein the tonicity agent is present in the formulation in an amount of about 5 mM to about 500 mM.

29. The formulation according to claim 27, wherein the tonicity agents are selected from the group consisting of sodium chloride, potassium chloride, glycerin, amino acids, sugars, as well as combinations thereof.

30. The formulation according to of claim 1, which can be administered by intravenous (i.v.) or subcutaneous (s.c.) or any other parenteral administration.

31. The liquid formulation of claim 2 which comprises:
about 1 to about 200 mg/mL Abeta antibody,
0.04% Tween 20 w/v,
20 mM L-histidine,
250 mM Sucrose,
at pH 5.5;
or
37.5 mg/mL Abeta antibody,
0.02% Tween 20 w/v,
10 mM L-histidine,
125 mM Sucrose,
at pH 5.5;
or
37.5 mg/mL Abeta antibody,
0.01% Tween 20 w/v,
10 mM L-histidine,
125 mM Sucrose,
at pH 5.5;
or
7.5 mg/mL Abeta antibody,
0.04% Tween 20 w/v,
20 mM L-histidine,
250 mM Sucrose,
at pH 5.5;
or
7.5 mg/mL Abeta antibody,
0.02% Tween 20 w/v,
10 mM L-histidine,
125 mM Sucrose,
at pH 5.5;
or
37.5 mg/mL Abeta antibody,
0.02% Tween 20 w/v,
10 mM L-histidine,
125 mM Trehalose,
at pH 5.5;
or
37.5 mg/mL Abeta antibody,
0.01% Tween 20 w/v,
10 mM L-histidine,
125 mM Trehalose,
at pH 5.5;
or
75 mg/mL Abeta antibody,
0.02% Tween 20 w/v,
20 mM L-histidine,
250 mM Trehalose,
at pH 5.5;
or
75 mg/mL Abeta antibody,
0.02% Tween 20 w/v,
20 mM L-histidine,
250 mM Mannitol,
at pH 5.5;
or
75 mg/mL Abeta antibody,
0.02% Tween 20 w/v,
20 mM L-histidine,
140 mM Sodium chloride,
at pH 5.5;
or
150 mg/mL Abeta antibody,
0.02% Tween 20 w/v,
20 mM L-histidine,
250 mM Trehalose,
at pH 5.5;
or
150 mg/mL Abeta antibody,
0.02% Tween 20 w/v,
20 mM L-histidine,
250 mM Mannitol,
at pH 5.5;
or
150 mg/mL Abeta antibody,
0.02% Tween 20 w/v,
20 mM L-histidine,
140 mM Sodium chloride,
at pH 5.5;
or
10 mg/mL Abeta antibody,
0.01% Tween 20 w/v,
20 mM L-histidine,
140 mM Sodium chloride,
at pH 5.5.

32. The lyophilized formulation of claim 3 which comprises:
about 1 to about 200 mg/mL Abeta antibody,
0.04% Tween 20 w/v,
20 mM L-histidine,
250 mM Sucrose,
at pH 5.5;
or
75 mg/mL Abeta antibody,
0.04% Tween 20 w/v,
20 mM L-histidine,
250 mM Sucrose,
at pH 5.5;
or
75 mg/mL Abeta antibody,
0.02% Tween 20 w/v,
20 mM L-histidine,
250 mM Sucrose,
at pH 5.5;
or
15 mg/mL Abeta antibody,
0.04% Tween 20 w/v,
20 mM L-histidine,
250 mM Sucrose,
at pH 5.5;
or
75 mg/mL Abeta antibody,
0.04% Tween 20 w/v,
20 mM L-histidine,
250 mM Trehalose,
at pH 5.5;
or
75 mg/mL Abeta antibody,
0.02% Tween 20 w/v,
20 mM L-histidine,
250 mM Trehalose,
at pH 5.5;
or
20 mg/mL Abeta antibody,
0.011% Tween 20 w/v,
5.3 mM L-histidine,
66.7 mM Sucrose,
at pH 5.5.

33. The liquid formulation of claim 2 or 31 which comprises:
10 mg/mL Abeta antibody,
0.01% Tween 20 w/v,
20 mM L-histidine,
140 mM Sodium chloride,
at pH 5.5.

34. The lyophilized formulation of claim 3 or 32 which comprises:
75 mg/mL Abeta antibody,
0.04% Tween 20 w/v,
20 mM L-histidine,
250 mM Sucrose,
at pH 5.5.

35. The lyophilized formulation of claim 3 or 32 which comprises:
20 mg/mL Abeta antibody,
0.011% Tween 20 w/v,
5.3 mM L-histidine,
66.7 mM Sucrose,
at pH 5.5.

36. The formulation according to claim 1, wherein the Abeta antibody comprises at least one antigen binding site comprising a glycosylated asparagine (Asn) in the variable region of the heavy chain (V_{H}).

37. The formulation according to claim 1, wherein the Abeta antibody is a defined mixture of
(a) Abeta antibody, wherein one of the antigen binding sites comprises a glycosylated asparagine (Asn) in the variable region of the heavy chain (V_{H}); and
(b) Abeta antibody, wherein both antigen binding sites comprise a glycosylated asparagine (Asn) in the variable region of the heavy chain (V_{H});
and which is free of or comprises to a very low extent Abeta antibody, wherein none of the antigen binding site comprises a glycosylated asparagine (Asn) in the variable region of the heavy chain (V_{H}).

38. The formulation according to claim 36 or 37, wherein the glycosylated asparagine (Asn) in the variable region of the heavy chain (V_{H}) is a glycosylated asparagine (Asn) in the CDR-2 region of the heavy chain (V_{H}).

39. The formulation according to claim 1, wherein the Abeta antibody comprises a heavy chain as defined in SEQ ID NO: 1 and a light chain as defined in SEQ ID NO: 2.

40. Use of a formulation according to claim 1 for the preparation of a medicament useful for treating Alzheimer's disease.

41. The invention as described hereinabove.
